(19) United States
(12) Patent Application Publication
(10) Pub. No.: US 2026/0097020 A1
(43) Pub. Date: Apr. 9, 2026

(54) THERAPEUTIC USE OF AROMATASE INHIBITORS AND POLY (ADP-RIBOSE) POLYMERASE (PARP) INHIBITORS FOR THE TREATMENT OF MALIGNANCIES

(71) Applicant: University of Cincinnati, Cincinnati, OH (US)

(72) Inventors: Pankaj B. Desai, Cincinnati, OH (US); Aniruddha S. Karve, Cincinnati, OH (US); Bhavesh B. Gabani, Cincinnati, OH (US); Sidharth N. Gadgil, Cincinnati, OH (US); Gary A. Gudelsky, Cincinnati, OH (US)

(21) Appl. No.: 19/345,169

(22) Filed: Sep. 30, 2025

Related U.S. Application Data
(60) Provisional application No. 63/701,054, filed on Sep. 30, 2024.

(51) Int. Cl. A61K 31/4196; A61K 31/4184; A61K 31/454; A61K 31/495; A61K 31/502; A61K 31/551; A61P 35/00

(52) U.S. Cl. CPC A61K 31/4196; A61K 31/4184; A61K 31/454; A61K 31/495; A61K 31/502; A61K 31/551; A61P 35/00

(57) ABSTRACT

Methods for treating gliomas, such as glioblastoma, including administering an aromatase inhibitor, such as letrozole, to a subject suffering from a glioma. The method may further include administering the aromatase inhibitor in combination with a poly-ADP ribose polymerase inhibitor, such as olaparib, pamiparib, veliparib or niraparib.

### CROSS-REFERENCE TO RELATED APPLICATIONS

This application claims priority to, and the benefit of the filing date of, U.S. Provisional Patent Application No. 63/701,054, filed Sep. 30, 2024, which is hereby incorporated by reference herein in its entirety.

### STATEMENT REGARDING FEDERALLY FUNDED RESEARCH OR DEVELOPMENT

This invention was made with government support under NS128232 awarded by the National Institutes of Health. The government has certain rights in the invention.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Glioblastoma (GBM) is one of the most aggressive and infiltrative brain malignancies. Despite significant advances in the multiple approaches for treatment of GBM, the median survival of patients has remained <15 months.

In view of the fact that prior and ongoing research has been unable to improve the prognosis for patients afflicted with GBM, it is clear that new treatments are needed.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

As noted above, prior and ongoing research has been unable to improve the prognosis for patients afflicted with GBM—and so, it is clear that new treatments are needed. To that end, research from the present inventors' lab has shown that aromatase (CYP19A1), an enzyme that catalyzes in situ production of estrogens, is markedly elevated in GBM tissues. Additionally, letrozole (LTZ), an aromatase inhibitor with extensive record of efficacious use in breast cancer, exhibited significant activity against GBM in preclinical studies. Moreover, in a phase 0/1 clinical trial in which recurrent GBM patients received LTZ (2.5-15 mg, N=18 subjects) prior to surgical resection, analyses of the excised tumor revealed that LTZ readily traverses the BBB in humans. RNA-seq analysis showed significant downregulation of oncogenes driving cell proliferation markers and upregulation of tumor suppression markers (reduced DNA damage repair capacity) in a LTZ dose-dependent manner.

In view of the present inventors' research, one aspect of the present invention is directed to use of LTZ as an anti-GBM drug. Another aspect of the present invention is directed to therapeutic combinations of LTZ with DNA-damage modulators such as, poly (ADP-ribose) polymerase inhibitors (PARPi)s.

Thus, one aspect of the invention is directed to a method comprising administering an aromatase inhibitor to a subject suffering from a glioma (such as a malignant glioma, an example of which is glioblastoma). One non-limiting example of such an aromatase inhibitor is letrozole.

Another aspect of the present invention is directed to a method of administering a combination of an aromatase inhibitor and a poly-ADP ribose polymerase inhibitor to a subject suffering from a glioma (such as a malignant glioma, an example of which is glioblastoma). One non-limiting example of such an aromatase inhibitor is letrozole. Examples of poly-ADP ribose polymerase inhibitors include, but are not limited to, olaparib, pamiparib, veliparib and niraparib.

Another aspect of the present invention is directed to a combination of an aromatase inhibitor and a poly-ADP ribose polymerase inhibitor. Such combination may be administered to a subject suffering from a glioma (such as a malignant glioma, an example of which is glioblastoma). The aromatase inhibitor and poly-ADP ribose polymerase inhibitor may be administered as a single composition. Alternatively, the aromatase inhibitor and a poly-ADP ribose polymerase inhibitor may be administered separately (i.e., not contained within a single composition) but combined as part of the same regimen. One non-limiting example of such an aromatase inhibitor is letrozole. Examples of poly-ADP ribose polymerase inhibitors include, but are not limited to, olaparib, pamiparib, veliparib and niraparib.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which are incorporated in and constitute a part of this specification, illustrate embodiments of the invention and, together with the general description of the invention given above and the detailed description of the embodiments given below, serve to explain the principles of the present invention.

FIG. 1A is a series of four graphs (shown in panels A-D) showing the cytotoxic effect of LTZ combined with various PARPis (niraparib in panel A, veliparib in panel B, pamiparib in panel C, and olaparib in panel C)±0.05 μM LTZ.

FIG. 1B is a series of microphotographs showing neurosphere growth inhibition with pamiparib±0.05 μM LTZ in G75 gfp luc cells.

FIG. 2 includes a graph showing the results of intracranial microdialysis for determining brain ECF and plasma PK profile for pamiparib in CD-IGS rats.

FIG. 3 includes two graphs showing the results of in vitro and in vivo metrics-based assessments of combination agents.

FIG. 4 includes a series of graphs in panels A, B, C, and D, showing plasma and brain PK profiles of various PARPis (pamiparib in panel A, niraparib in panel B, velparib in panel C, and Olaparib in panel D).

FIG. 5 includes a series of graphs and microphotographs showing data for percentage of cell survival as a function of drug levels.

FIG. 6 includes a series of graphs showing that pamiparib at concentrations up to 1 M had no noticeable induction of γ-H2AX in four patient-derived GBM lines (G43, G75, JHH-136, and G76).

FIG. 7 includes a series of graphs showing that cells, when treated with pamiparib, had significant induction of apoptosis at concentrations below 1 μM.

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

As noted above, prior and ongoing research has been unable to improve the prognosis for patients afflicted with GBM. Coupled with that: (1) research from the present inventors' lab has shown that aromatase (CYP19A1), an enzyme that catalyzes in situ production of estrogens, is markedly elevated in GBM tissues, (2) letrozole (LTZ), an aromatase inhibitor with extensive record of efficacious use in breast cancer, exhibited significant activity against GBM in preclinical studies, and (3) in a phase 0/1 clinical trial in which recurrent GBM patients received LTZ (2.5-15 mg, N=18 subjects) prior to surgical resection, analyses of the excised tumor revealed that LTZ readily traverses the BBB in humans. RNA-seq analysis showed significant downregulation of oncogenes driving cell proliferation markers and upregulation of tumor suppression markers (reduced DNA damage repair capacity) in a LTZ dose-dependent manner.

And so, one aspect of the invention is directed to a method comprising administering an aromatase inhibitor to a subject suffering from a glioma (such as a malignant glioma, an example of which is glioblastoma). The subject may have been diagnosed with a glioma (such as a malignant glioma, such as glioblastoma). One non-limiting example involves using letrozole (LTZ), a third-generation aromatase inhibitor, as a novel therapeutic for the treatment of malignant gliomas including glioblastoma (GBM). It synergistically enhances the efficacy of the DNA alkylating agent temozolomide, one of the few approved chemotherapeutics for GBM, against patient-derived GBM cells. Thus, another embodiment of this aspect of the present invention is directed to administering an aromatase inhibitor (such as letrozole) to a subject that is also administered temozolomide.

RNA-seq analysis of tumor from recurrent GBM patients treated with LTZ prior to surgery in a “window of opportunity” phase 0/1 dose escalation clinical trial revealed that LTZ downregulates genes encoding for DNA-damage repair proteins (BRCA2) in a dose dependent manner. This indicates that the combination of LTZ with targeted therapeutic agents that promote DNA damage results in synergistic effects against GBM. And so, another aspect (and embodiments) of the present invention are directed to a method of administering a combination of an aromatase inhibitor and a poly-ADP ribose polymerase inhibitor to a subject suffering from a glioma (such as a malignant glioma, an example of which is glioblastoma). One non-limiting example of such an aromatase inhibitor is letrozole. Examples of poly-ADP ribose polymerase inhibitors include, but are not limited to, olaparib, pamiparib, veliparib and niraparib. In one embodiment, the present invention uses poly-ADP ribose polymerase (PARP) inhibitors in combination with LTZ to treat GBM.

Another aspect of the present invention is directed to a combination of an aromatase inhibitor and a poly-ADP ribose polymerase inhibitor. Such combination may be administered to a subject suffering from a glioma (such as a malignant glioma, an example of which is glioblastoma). The aromatase inhibitor and poly-ADP ribose polymerase inhibitor may be administered as a single composition. Alternatively, the aromatase inhibitor and a poly-ADP ribose polymerase inhibitor may be administered separately (i.e., not contained within a single composition) but combined as part of the same regimen. One non-limiting example of such an aromatase inhibitor is letrozole. Examples of poly-ADP ribose polymerase inhibitors include, but are not limited to, olaparib, pamiparib, veliparib and niraparib.

### EXAMPLES

### Example 1

Neurosphere Growth and Cell Viability Assays: Employing patient-derived G43, G75 and JHH-136 GBM lines, the present inventors assessed the influence of PARP inhibitors (olaparib, pamiparib, veliparib and niraparib) in combination with LTZ on cell viability and neurosphere growth.

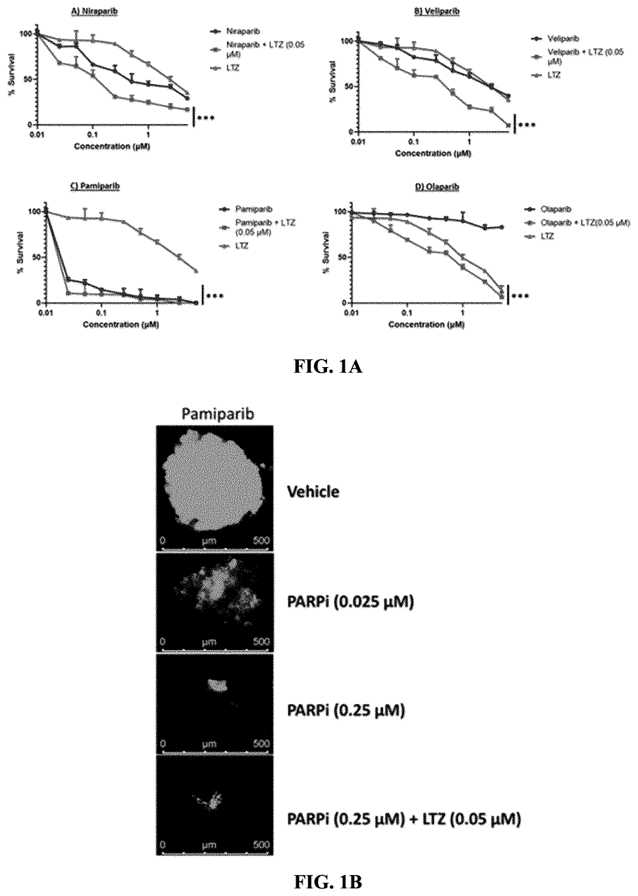

As shown in FIGS. 1A and 1B, and in Table 1 (below), LTZ (0.05 μM) markedly potentiated the cytotoxicity of each of the four PARPi and substantially lowered the IC₅₀ values.

TABLE 1
IC₅₀ values of LTZ and PARPi ± 0.05 μM LTZ; against G43, G75 and JHH-136 GBM cells

| | Treatment IC₅₀ | | | | | | | | | | | | | | |
|---|---|---|---|---|---|---|---|---|---|---|---|---|---|---|---|
| | | Nimparib | | Veliparib | | Pamiparib | | Olaparib | | Ribociclib | | Palbociclib | | Abemaciclib | |
| | | (μM) | | (μM) | | (μM) | | (μM) | | (μM) | | (μM) | | (μM) | |
| Cell | LTZ | (−) | (+) | (−) | (+) | (−) | (+) | (−) | (+) | (−) | (+) | (−) | (+) | (−) | (+) |
| line | (μM) | LTZ | LTZ | LTZ | LTZ | LTZ | LTZ | LTZ | LTZ | LTZ | LTZ | LTZ | LTZ | LTZ | LTZ |
| G43 | 0.653 | 2.39 | 0.17 | 8.39 | 0.293 | 0.26 | 0.08 | 11.43 | 0.85 | 14.39 | 0.8 | >100 | >100 | 15.1 | 5.8 |
| G75 | 1.235 | 0.819 | 0.124 | 2.13 | 0.615 | 0.16 | 0.012 | 19.35 | 0.591 | 0.418 | 0.154 | 9.6 | 15.11 | 0.19 | 0.1 |
| JHH- | 2.13 | 1.52 | 0.385 | 6.18 | 1.01 | 0.588 | 0.127 | 15.18 | 2.8 | 4.839 | 1.816 | >100 | >100 | 18.8 | 21.2 |
| 136 | | | | | | | | | | | | | | | |

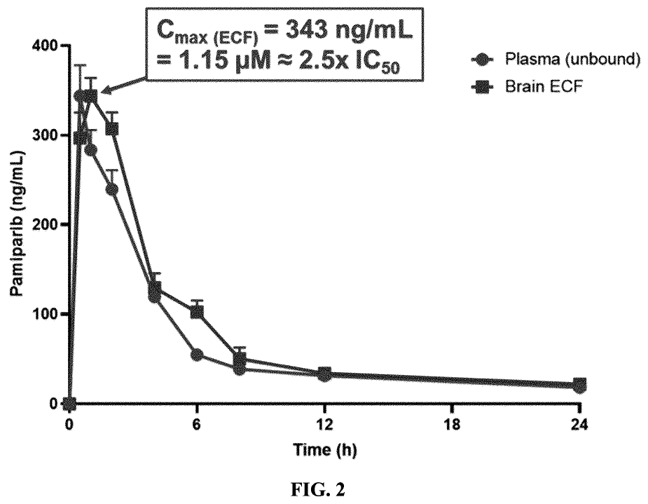

Brain Extracellular Fluid (ECF) and Plasma Pharmacokinetics: Referring to FIG. 2, the present inventors then utilized male CD-IGS rats (n=6; body wt., 250 g) to assess plasma and brain extracellular fluid (ECF) pharmacokinetics (PK) of PARP inhibitors using cerebral microdialysis. The IC₅₀ values for PARP inhibitors in these assays ranged from 0.06 to 11.31 μM. Treatment in combination with LTZ (0.05 μM), a non-cytotoxic concentration of LTZ in these lines, significantly decreased the IC₅₀ values for PARP inhibitors (0.012 to 0.51 μM).

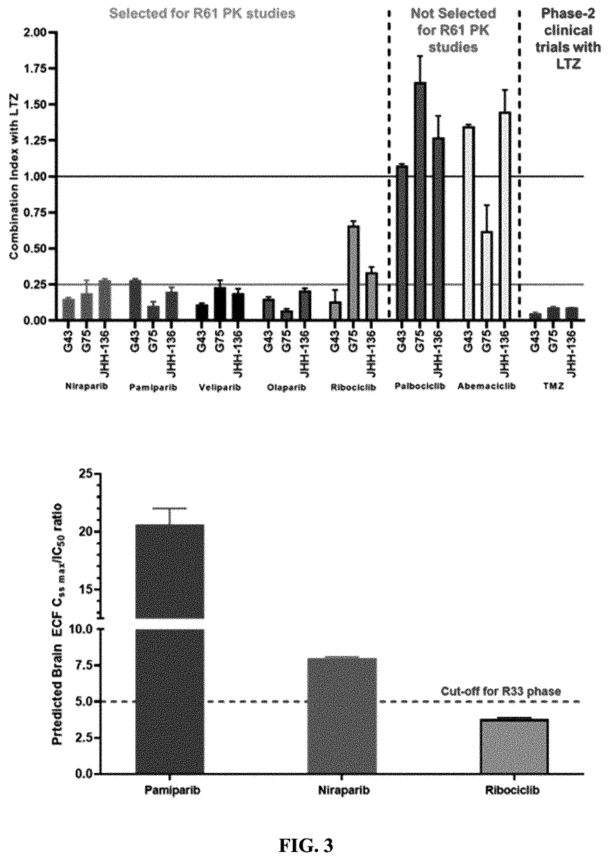

Pharmacodynamics of Drug Interaction: The present inventors then employed Combination Index Analysis to assess whether the combinations were additive, synergistic or antagonistic. The Combination Index analysis indicated that this combination was strongly synergistic (see FIG. 3). Furthermore, and referring to both FIG. 3 and Table 2 (below), the plasma and brain ECF PK analyses suggested that PARP inhibitors such as pamiparib and niraparib exhibit facile transport across the BBB barrier as reflected in K_{p,uu,brain} (ratio of AUC_{ecf}/AUC_{plasma, unbound}) values of 0.98 and 0.45, respectively.

TABLE 2
Plasma and Brain ECF PK of Niraparib, Pamiparib, and Ribociclib in CD-IGS rats

| | Drug | | | | | | | | | | | |
|---|---|---|---|---|---|---|---|---|---|---|---|---|
| | Niraparib | | | | Pamiparib | | | | Ribociclib | | | |
| PK | Plasma | | | | Plasma | | | | Plasma | | | |
| Parameter | (unbound) | | Brain ECF | | (unbound) | | Brain ECF | | (unbound) | | Brain ECF | |
| Cₘₐₓ | 375 | ng/mL | 113 | ng/mL | 343.83 | ng/mL | 302.25 | ng/mL | 289.4 | ng/mL | 31.2 | ng/mL |
| Tₘₐₓ | 0.67 | h | 2 | h | 0.58 | h | 1.07 | h | 4 | h | 4 | h |
| Elimination | 3.8 | h | 5.6 | h | 9.7 | h | 4.7 | h | 3.1 | h | 1.6 | h |
| half-life | | | | | | | | | | | | |
| AUC_{(0-inf)} | 2383.3 | h · ng/mL | 1088 | h · ng/mL | 1473 | h · ng/mL | 1488 | h · ng/mL | 1300 | h · ng/mL | 173 | h · ng/mL |

| | | | |
|---|---|---|---|
| K_{p, uu, brain} | 0.45 | 1.01 | 0.13 |

Metrics-Based Go/No Go Decision: This integrated approach provided global score for advancing the combination to next step(s).

Overall, these results provide a strong foundation for pursuing further development of combination therapy of GBM with LTZ and PARP inhibitors. A careful assessment of the clinical efficacy and safety profile of these agents will further guide shortlisting combination of LTZ for pre-clinical and clinical development.

### Example 2

As described above, glioblastoma (GBM) is the most common and aggressive primary malignant brain tumor in adults, with an incidence of 3.2 per 100,000 population annually and a dismal prognosis [1]. Standard-of-care therapy, consisting of maximal safe resection followed by concomitant radiotherapy and temozolomide chemotherapy, yields a median overall survival of approximately 15 months, and long-term survival remains rare (<7% at five years) [2, 3]. Multiple factors contribute to therapeutic failure, including intrinsic and acquired resistance mechanisms, profound intratumoral heterogeneity, and the diffuse, infiltrative nature of GBM that precludes complete surgical resection. At the molecular level, GBM cells are adept at engaging DNA damage response (DDR) pathways, particularly homologous recombination repair (HRR) and base excision repair (BER), to survive cytotoxic insults, underscoring the need for innovative approaches targeting tumor DNA repair vulnerabilities.

Aromatase inhibitors (AIs) represent one such therapeutic class with potential for repurposing in neuro-oncology. Aromatase (CYP19A1) catalyzes the rate-limiting step in the biosynthesis of estrogens by converting androgens into estrogens in peripheral tissues. Third-generation AIs, including letrozole (LTZ), anastrozole, and exemestane, are highly selective and potent inhibitors of aromatase, developed to suppress estrogen-driven proliferation in postmenopausal estrogen receptor (ER)-positive breast cancer [4, 5]. LTZ, a non-steroidal reversible inhibitor, is distinguished by near-complete systemic estrogen suppression and a favorable safety profile in long-term use. While initially conceived for hormone-sensitive breast cancer, emerging data demonstrate that estrogens may influence glioma biology via estrogen receptor signaling, cell cycle regulation, and DNA repair pathways. The present inventors' work revealed that LTZ readily crosses the blood-brain barrier (BBB), accumulates in tumor tissue, and exerts anti-glioma effects through inhibition of cell proliferation and potentiation of temozolomide efficacy [6, 7]. Further, a Phase 0/1 clinical trial in recurrent GBM established LTZ's CNS penetrance, safety, and pharmacodynamic activity. RNA-sequencing of LTZ-exposed tumor tissue uncovered dose-dependent downregulation of BRCA2 and other HRR-associated genes [8] implicating LTZ as a functional modulator of DNA repair capacity and raising the possibility of exploiting synthetic lethality.

PARPi constitute a mechanistically complementary class of agents that directly target DDR. PARP1 and PARP2 enzymes orchestrate BER by sensing single-strand DNA breaks and recruiting repair machinery through poly(ADP-ribosyl)ation. Pharmacological inhibition of PARP traps the enzyme on damaged DNA and stalls replication forks, converting single-strand breaks into cytotoxic double-strand breaks. In HRR-deficient backgrounds, such as BRCA1/2-mutated tumors, PARPi-induced DNA lesions cannot be accurately repaired, culminating in cell death—a paradigm of synthetic lethality [9]. Clinically, PARPi have reshaped cancer therapy: olaparib, niraparib, rucaparib, and talazoparib are FDA-approved for ovarian, breast, pancreatic, and prostate cancers harboring HRR defects [10-12]. Veliparib and pamiparib, though not yet globally approved, are in late-stage trials. Beyond their established role in gynecologic and prostate malignancies, PARPi exhibit radiosensitizing and chemosensitizing activity in glioma models [13, 14]. However, translational hurdles include poor BBB permeability for certain agents (e.g., olaparib), variability in CNS penetration, and dose-limiting hematologic toxicities that constrain therapeutic indices.

The present inventors have now developed, among other aspects of invention disclosed herein, a combination therapy in malignancies such as GBM using aromatase inhibitors and PARPis. Mechanistically, LTZ-mediated BRCA2 downregulation may sensitize otherwise HRR-proficient GBM cells to PARPi-induced DNA damage, enhancing tumor cell kill. Furthermore, LTZ's metabolic disposition, largely via CYP2A6, reduces the likelihood of clinically meaningful pharmacokinetic drug-drug interactions with PARPi, which are predominantly substrates of CYP3A4 or carboxylesterases [15]. Importantly, intracerebral microdialysis studies in rodents have demonstrated that select PARPi, including pamiparib, niraparib, and veliparib, achieve measurable concentrations in brain extracellular fluid, aligning with cytotoxic thresholds in preclinical assays, whereas olaparib exhibits limited brain penetration.

Taken together, the combination of LTZ and PARPi leverages two mechanistically distinct but convergent strategies to disrupt tumor DNA repair: pharmacological estrogen suppression leading to HRR downregulation, and enzymatic blockade of BER leading to replication stress. This dual assault on GBM's robust DNA repair machinery has strong translational potential. In this Example 2, the present inventors provide data regarding the therapeutic synergy of LTZ with four clinically advanced PARPi-olaparib, niraparib, veliparib, and pamiparib-across patient-derived GBM models. The present inventors integrate cytotoxicity and apoptosis assays with pharmacokinetic profiling via intracerebral microdialysis to assess CNS penetrance, thereby providing preclinical and translational evidence to justify clinical exploration of LTZ-PARPi combinations in GBM.

### Materials and Methods:

### Cell Lines and Reagents:

Patient-derived GBM cells, G43, G75, and G76 were kindly provided by Dr. Jann Sarkaria, (Mayo Clinic Brain Tumor Patient-Derived Xenograft National Resource) [16] JHH-136 cells were obtained from Department of Cancer Biology, University of Cincinnati (originally obtained from Dr. Gregory Riggins, John Hopkins University) [17]. Neural stem cell culture medium, Gibco™ StemPro™ NSC SFM (Catalog #A1050901), penicillin (50 U/mL), and streptomycin (50 mg/mL), letrozole (99% pure) and CyQuant™ LDH assay kit were purchased from Fisher Scientific. Olaparib, veliparib, niraparib and pamiparib were purchased from Selleck Chemicals Inc. Rabbit monoclonal anti-gamma H2A.X (phospho S139) antibody [9F3](Catalog #ab26350) was obtained from Abcam Inc. Annexin V apoptosis assay kit was purchased from Abcam Inc. Dialysis membrane (MW cutoff, 13,000 Da and outside diameter, 210 m) was purchased from Spectrum Laboratories, CA. Cannulation materials for the implantation of venous catheters were purchased from Plastics One Inc. (Roanoke, VA) and VWR scientific (Batavia, IL). BD Microtainer® K2EDTA coated tubes were purchased from VWR Scientific (Philadelphia, PA). Rapid Equilibrium Dialysis Device Single-Use Protein binding kit and phosphate-buffered saline were purchased from Thermo Scientific™

### Animals:

Jugular vein cannulated (JvC) Sprague-Dawley (SD) rats (Male, body weight: 200-250 g, Age: 6 Weeks) were purchased from Charles River Laboratories (Wilmington, MA). Animals were housed individually in a room maintained at 22° C. with The University of Cincinnati Institutional Animal Care and Use Committee (IACUC) approved standards for relative humidity and light/dark cycle. Access to water and standard laboratory chow ad libitum was also provided. The animal handling and experimental protocol was approved by the University of Cincinnati IACUC (Protocol No. 23-06-05-01).

### Cell Culture and Maintenance:

Patient-derived GBM cells were grown and maintained as described in the present inventors' previous publication (Vaubel et al., 2018). Briefly, the cells were grown in T25 flasks containing neural stem cell growth medium supplemented with NSC growth supplement, hEFG, hFGF and 10% penicillin & streptomycin and maintained in a 5% CO₂ incubator at 37° C. Use of any serum containing material was avoided. The confluent single cell suspensions were used for cell counting using a hemocytometer and trypan blue exclusion and seeded into appropriate plates at appropriate densities for further assays.

### Microdialysis Surgery for Assessment of PK in Rats:

Microdialysis studies in rats were performed to determine brain ECF PK of PARPi as described in the present inventors' previous publications (Desai et al., 2022, Dave et al., 2013, Arora et al., 2019). Briefly, SD-JvC-rats (N=6) were intracranially implanted with stainless steel guide cannula under ketamine/xylazine (70/6 mg/kg i.p.) anesthesia 24 h before the experiment. A concentric dialysis probe was inserted through the guide cannula into the striatum on the day of the experiment. The coordinates for the guide cannula were 1.2 mm anteroposterior and 3.1 mm lateral from bregma, according to the stereotaxic atlas (Paxinos and Watson, 2005). The active portion of the membrane for the striatum was 4.5 mm. The probes were connected to an infusion pump set to deliver modified Dulbecco's phosphate buffered saline at a rate of 2.0 L/min.

### Drug Administration and Sampling Protocol:

PARPi 100 mg were dissolved in 0.5 mL DMSO and further diluted to a concentration of 20 mg/mL in a solvent system consisting of propylene glycol (40%), ethanol (5%) and injectable saline (55%) and filtered using a 0.2 micron filter prior to injection. After an equilibration period of 30 min, each PARPi (20 mg/kg) was administered separately via intraperitoneal (i.p.) route. Dialysate (via probe outlet tubing) and blood (via jugular catheter) samples were collected at 0, 0.5, 1, 2, 4, 6, 8, 12 and 24 h after administration. All study samples were stored at −80° C. prior to analysis.

### Assessment of In Vitro Recovery of PARPi Using Retrodialysis:

Dialysis buffer containing a known concentration of PARPi (20 μM) was perfused through the probe. Probes were equilibrated for 1 h, followed by sample collection at every 30 min interval for 3 h. The % loss of PARPi was determined as an estimate of in vitro recovery.

### Bioanalysis and PK Assessment:

PARPi from the samples was extracted using liquid-liquid extraction method with methyl tert-butyl ether as an extraction solvent. The concentrations of the PARPi in plasma and brain ECF were analyzed using LC-MS/MS. Compartmental analysis for estimation of important PK parameters, Cₘₐₓ, tₘₐₓ, AUC₀₋₁₂ₕ, t_{1/2}, and k_{p,u,u,brain} was performed using Phoenix® WinNonlin version 8.4.3 (Certara).

### Cell Viability Assays:

The cytotoxic effects of PARPi, LTZ and combinations of the two agents were evaluated using the CyQuant™ Lactate Dehydrogenase (LDH) assay (ThermoFisher Scientific). Cells at the seeding density of 1.5×10⁴/well, were plated in 96 well plates in the above-mentioned serum-free medium. Drug treatments were added 24 hours after seeding with treatment concentrations ranging as follows, PARPi (0-20 μM), LTZ (0-20 μM), or PARPi (0-20 μM)+LTZ (50 nM). Dimethyl sulfoxide (0.1%) was used as a vehicle control. The PARPi were then rank-ordered based on potency in combination with LTZ and the BBB permeability based on k_{p,u,u,brain}, and the most potent and BBB permeable PARPi was then selected for further functional and mechanistic evaluation.

### Limiting Dilution Assay (LDA) for Assessment of Functional Activity of Shortlisted PARPi and LTZ:

Single-cell suspensions were prepared and plated in ultra-low attachment 96-well plates at serial dilutions (e.g., 1,000; 300; 100; 30; 15; and 5 cells/well) in NSC medium. Cells were treated with shortlisted PARPi (ranges centered on sub-IC₅₀ to IC₅₀ obtained from LDH), and PARPi+LTZ (50 nM). Plates were incubated for 10 days. Wells were scored for neurosphere formation using a predefined diameter threshold by blinded evaluators. Limiting dilution plots and stem cell frequencies were generated using online software provided by the Walter and Eliza Hall Institute of Medical Research [18]

### Assessment of DNA-Damage Induced by PARPi+LTZ Combination:

DNA-damage induced by combination of the shortlisted PARPi and LTZ was measured using flow cytometric analysis for induction of γH2A.x as described in the present inventors' previously published study [7, 19]. Briefly, 1×10⁶ cells were seeded in a 6-well plate and treated with the shortlisted PARPi (0-10 μM), LTZ (0-10 μM) or PARPi (0-10 μM)+LTZ (50 nM) for 5 hours. Cell pellets were collected after centrifugation at 2000×g for 5 minutes. Cells were then washed with ice-cold PBS and fixed and permeabilized with 1:1 mixture of methanol and NP40 (300 L). Primary unconjugated antibody for γH2A.x (100 L of 1:250 dilution) was added to the cell pellets after permeabilization and incubated for 20 minutes at room temperature. Cells were then washed with 600 μL of 1:1 methanol and NP40 mixture. Secondary AlexaFluor® 488 antibody was added to the cells at a dilution of 1:1000 and incubated at room temperature in the dark for 20 min. This was followed by a wash step as described earlier and the cells were re-suspended in 1×PBS and subjected to flow cytometric analysis at an excitation wavelength of (488 nm) employing Beckman CytoFlex flow cytometer. Gating was done using Human IgG-isotype control as outlined in the manufacturer's protocol. The scatter plots were analyzed using CytExpert version 2.5, to assess the percentage of cells positive for γH2A.x versus the isotype control.

### Measurement of Apoptosis Induction:

Apoptosis induction was measured using Annexin V kit obtained from Abcam Inc. Cells (1×10⁵) were seeded in a 12-well plate and treated with PARPi (0-10 μM), LTZ (0-10 μM) or PARPi (0-10 μM)+LTZ (50 nM) for 48 hours. This was followed by incubation of cells with 1× final mixture of propidium iodide (PI), annexin V and binding buffer for 10 minutes in dark at room temperature. The binding of annexin V to the cell surface and PI to the cell core was then assessed using Leica DMi8 Widefield Fluorescence microscope.

### Statistical Analyses:

Data are presented as mean±standard deviation unless stated. IC₅₀ values were estimated by nonlinear regression. Multiple comparisons for cytotoxicity were assessed using t-tests with appropriate correction or ANOVA as applicable (GraphPad Prism v10) Differences between the groups were considered significant with P values P≥0.05 (ns, nonsignificant); *, P<0.05; **, P<0.01; ***, P<0.001; ****, P<0.0001). Pharmacokinetic parameters were derived using noncompartmental and compartmental methods (Phoenix WinNonlin v8.4.3). Statistical significance was predefined at two-sided α=0.05.

### Results:

### Plasma and Brain ECF Pharmacokinetics of PARPi in SD-Rats:

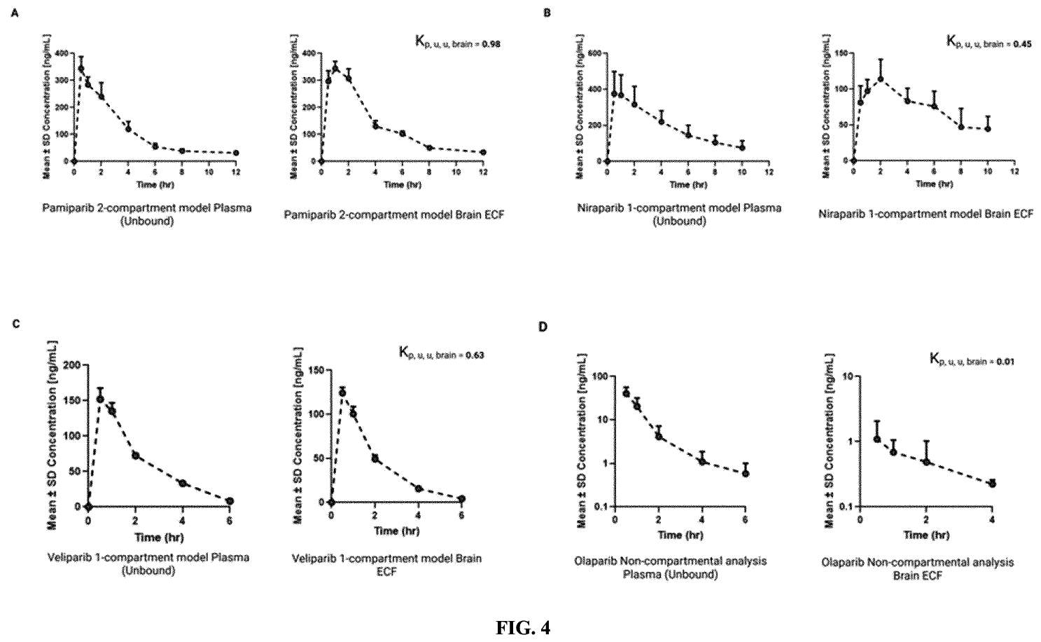

The plasma and brain PK profiles of PARPi are shown in FIG. 4 and PK data are provided in Table 3. To assess the brain partitioning of PARPi, the present inventors employed intracerebral microdialysis to simultaneously collect the brain ECF samples and blood samples from the JvC. This facilitated discernment of the time course, the rate, and the extent of BBB permeability of the PARPi. Following intraperitoneal administration, the peak unbound plasma drug concentrations (Cₘₐₓ) of the PARPi were observed to be as follows: pamiparib (344 ng/mL); niraparib (375 ng/mL); olaparib (30 ng/mL) and veliparib (157 ng/mL). The time to achieve Cₘₐₓ(Tₘₐₓ) was between 0.5 and 1 h post administration for all four PARPi. Brain ECF Cₘₐₓ for all four PARPi was observed to be as follows: pamiparib (302 ng/mL); niraparib (113 ng/mL); olaparib (<1 ng/mL) and veliparib (105 ng/mL) (Table 4). The half-life of all the PARPi in brain ECF and plasma were similar, denoting low accumulation in the brain (Table 4). The ratio of brain ECF AUC₀₋₁₂ₕ to the unbound plasma AUC₀₋₁₂ₕ (k_{p,uu,brain}) were found to be as follows: pamiparib (0.98); niraparib (0.45); olaparib (<0.1) and veliparib (0.57). The k_{p,uu,brain} estimates for PARPi are also shown in Table 3.

TABLE 3
Plasma and Brain Pharmacokinetics of PARPi in SD rats (n = 6; M = 3, F = 3; age = 6 weeks)

| PK parameter | Pamiparib | | Niraparib | | Veliparib | | Olaparib | |
|---|---|---|---|---|---|---|---|---|
| C [symbol] | 344 ± 25 | ng/mL | 375 ± 64 | ng/mL | 157 ± 35 | ng/mL | 40.8 ± 8.9 | ng/mL |
| C [symbol] | 302 ± 13 | ng/mL | 113 ± 12 | ng/mL | 118 ± 15 | ng/mL | 0.5 ± 0.1 | ng/mL |
| AUC [symbol] | 1488 ± 110 | h · ng/mL | 2383 ± 253 | h · ng/mL | 1065 ± 369 | h · ng/mL | 612 ± 426 | h · ng/mL |
| AUC [symbol] | 1473 ± 80 | h · ng/mL | 1088 ± 220 | h · ng/mL | 697 ± 129 | h · ng/mL | 5.19 ± 3.2 | h · ng/mL |
| T_{1/2} | 9.7 | h | 3.8 | h | 6 | h | 1 | h |

| | | | | |
|---|---|---|---|---|
| K [symbol] | 0.98 | 0.46 | 0.63 | 0.01 |

[symbol]

indicates data missing or illegible when filed

TABLE 4
IC₅₀ and combination index values for LTZ, PARPis and a combination of PARPi
with non-cytotoxic concentrations of LTZ against patient-derived GBM lines.

| | | Olaparib | | | Veliparib | | |
|---|---|---|---|---|---|---|---|
| Cell | Olaparib | (μM) + | C.I. | Veliparib | (μM) + | C.I. | Niraparib |
| line | (μM) | LTZ (50 nM) | (IC₅₀) | (μM) | LTZ (50 nM) | (IC₅₀) | (μM) |
| G43 | 11.43 | 0.85 | 0.15 | 8.39 | 0.293 | 0.11 | 2.39 |
| G75- | 19.35 | 0.591 | 0.07 | 2.13 | 0.615 | 0.33 | 0.819 |
| gfp-luc | | | | | | | |
| JHH- | 15.18 | 2.8 | 0.21 | 6.18 | 1.01 | 0.341 | 1.52 |
| 136 | | | | | | | |
| G76 | 1.39 | 0.084 | 0.69 | 0.49 | 0.07 | 0.767 | 0.194 |

| | Niraparib | | | Pamiparib | | |
|---|---|---|---|---|---|---|
| Cell | (μM) + | C.I. | Pamiparib | (μM) + | C.I. | LTZ |
| line | LTZ (50 nM) | (IC₅₀) | (μM) | LTZ (50 nM) | (IC₅₀) | (μM) |
| G43 | 0.17 | 0.15 | 0.16 | 0.08 | 0.57 | 0.653 |
| G75- | 0.44 | 0.58 | 0.06 | 0.012 | 0.24 | 1.235 |
| gfp-luc | | | | | | |
| JHH- | 0.585 | 0.40 | 0.488 | 0.177 | 0.39 | 2.13 |
| 136 | | | | | | |
| G76 | 0.04 | 0.83 | 0.09 | 0.012 | 0.76 | 0.08 |

Evaluation of Cytotoxic Effects of Combinations of PARPi with LTZ and Shortlisting the Most Potent PARPi for Further Evaluation:

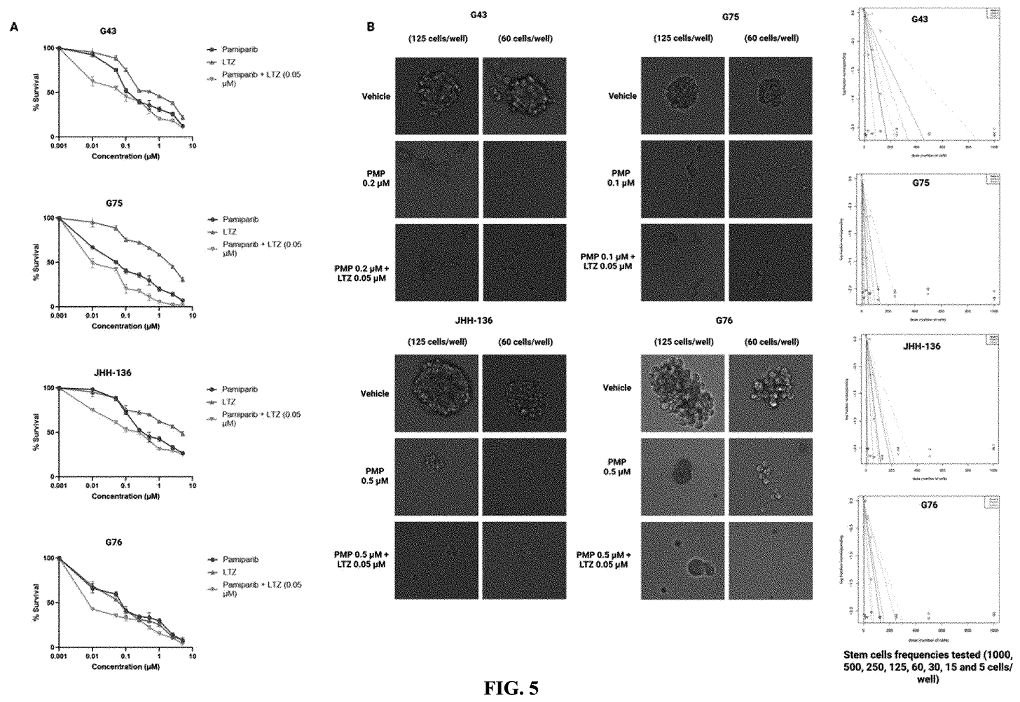

The cytotoxic effects of LTZ and PARPi on various patient-derived GBM lines were examined using LDH assay. Initially the cytotoxic effects of each compound were assessed individually. This was followed by evaluation of cytotoxic effects of combinations of PARPi with LTZ (50 nM), a relatively non-cytotoxic concentration. The data for percentage cell survival as function of drug levels are shown in FIG. 5 and the in vitro IC₅₀ determined using this data are shown in Table 4. All four lines were fairly sensitive to LTZ with IC₅₀ values ranging from 0.08 to 2.1 μM. From the PARPi used for this study, cells were reasonably responsive to treatment with pamiparib (IC₅₀ range: 0.09 to 0.48 μM) and niraparib (IC₅₀ range: 0.19 to 2.4 μM) as single agents. On the other hand the cells were found to be less responsive to treatment with olaparib (IC₅₀ range: 1.4 to 19.3 μM) and veliparib (IC₅₀ range: 0.5 to 8.4 μM). As shown in Table 3 and FIG. 4, combination of all four PARPi with LTZ (50 nM) significantly increased the cytotoxic effects of all four PARPi and the range of IC₅₀ values was significantly reduced across the panel of GBM lines. Based on the data obtained from the PK studies and the LDH assay, the PARPi were ranked for potency and BBB permeability, and pamiparib was observed to have adequate cytotoxicity profile and brain ECF exposure. As such, further mechanistic and functional assessments were performed using pamiparib as the shortlisted PARPi.

### LDA Results:

### Assessment of Induction of γH2A.x and Apoptosis by Combination of PARPi and LTZ:

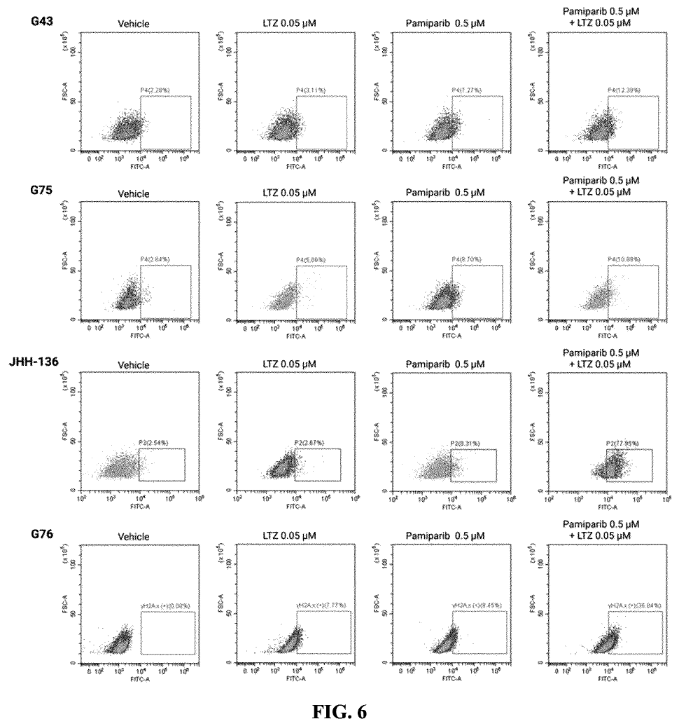

Since the present inventors demonstrated in the present inventors' previous study that LTZ imparts DNA-damage by induction of γH2A.x in GBM lines and mechanistically pamiparib should inhibit DNA-damage response resulting in cell death, the present inventors investigated the DNA-damaging and apoptotic effects of these compounds in patient-derived GBM lines. The present inventors measured γ-H2AX formation at 5 h after treatment with pamiparib and LTZ combinations. As shown in FIG. 6, pamiparib at concentrations up to 1 M had no noticeable induction of γ-H2AX in the patient-derived GBM lines. Treatment with LTZ as a single agent up to 1 μM considerably increased γ-H2AX levels in all four lines. Most importantly, LTZ (50 nM) when combined with pamiparib (0.05-1 μM) significantly increased the number of events positive for γ-H2AX.

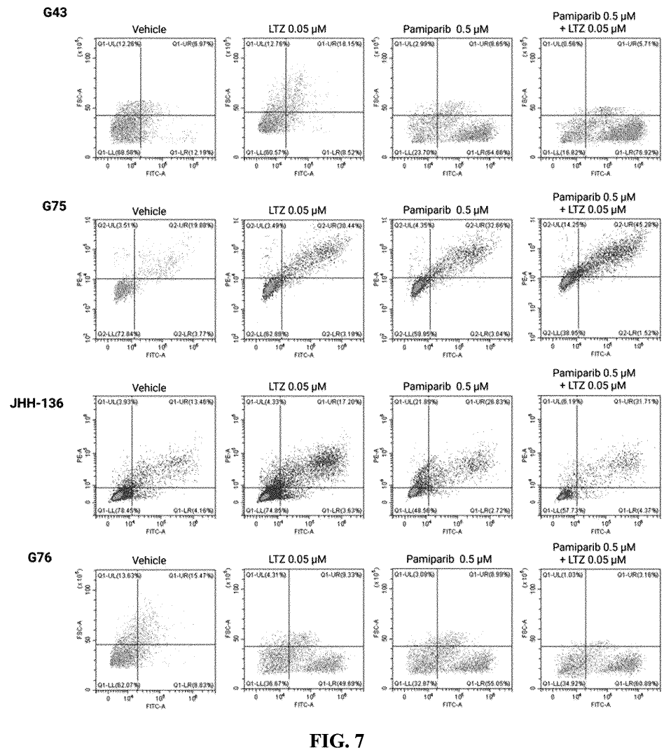

The effect of LTZ and pamiparib combinations on induction of apoptosis in GBM lines was also assessed using annexin V staining. Annexin V binds to phosphatidylserine on the outer part of the cell membrane demarcating the last step of apoptosis. The present inventors assessed the annexin V positive cells using fluorescence microscopy in GBM cells treated with PARPi and LTZ combinations. As shown in FIG. 7, cells when treated with pamiparib had significant induction of apoptosis at concentrations below 1 μM. Most importantly addition of LTZ at non-cytotoxic concentrations resulted in a significant increase in induction of apoptosis by pamiparib.

### DISCUSSION

The present inventors' previous work has demonstrated the in vitro efficacy of LTZ and TMZ combination against a panel of GBM lines. Additionally, the RNA-sequencing data from the tumor samples of patients enrolled in phase-0/1 “window of opportunity” clinical trial for LTZ highlights the key molecular pathways impacted by LTZ in a dose-dependent manner [7, 8]. These pathways include downregulation of genes encoding the DNA-damage repair proteins such as, BRCA2, RAD54L, and CHEK1. PARPi inhibit the DNA-damage repair through inhibition of PARP-1 and PARP-2, the proteins involved in induction of MGMT- and BER-mediated DNA-damage repair, in homologous recombination (HR)-deficient-BRCA 1/2-mutant cancers [20]. Hence, the present inventors' attempt in this study was to assess synergistic interactions between PARPi and LTZ against GBM and identify novel efficacious and brain-penetrant combination partners with LTZ as GBM therapeutics [6].

All the PARPi, used for this study, are either approved as the first line of treatment or are being evaluated in clinical trials, for variety of HR-deficient cancers, including ovarian cancer, triple-negative breast cancer and castration-resistant prostate cancer. Hence, these drugs have been extensively studied for their PK properties, drug-drug interactions and safety profile in clinical setting. While other PARPi such as, talazoparib and rucaparib are also approved for the treatment of HR-deficient cancers, these compounds are substrates of efflux transporters, ABCB1 and ABCG2, and hence have limited permeability across the BBB. On the other hand, PARPi used for this study are currently being evaluated clinically for their efficacy as anti-GBM therapeutics and hence, these PARPi were shortlisted as potential combination partners for LTZ. The present inventors' in vitro findings from this study support the hypothesis that LTZ potentiates cytotoxicity of PARPi in a panel of GBM lines with different genotypic and phenotypic characteristics (Table 5). The present inventors' studies further demonstrate that treatment of GBM lines with a combination of PARPi and LTZ results in significant DNA-damage, as quantified by the dose-dependent increase in γ-H2AX, and apoptosis, as quantified by the dose-dependent increase in Annexin V. The DNA-damaging and apoptotic effects of the combination were markedly enhanced as compared to treatment with these drugs used as single agents. Notably, relatively low concentrations of LTZ were sufficient to potentiate the effects of PARPi against GBM cells. Most significant cytotoxic effects of combination treatment were observed with pamiparib and niraparib. Furthermore, pamiparib was also found to be effective as a single agent with the IC₅₀ values ranging from (0.09 to 0.48 μM) against the panel of GBM lines used for this study and the IC₅₀ values of pamiparib significantly decreased in combination with 50 nM of LTZ (IC₅₀ range: 0.012 to 0.18 μM). Efficacy of other PARPi was lower than pamiparib as single agents, but it was enhanced in combination with LTZ (Table 3).

TABLE 5
Patient-Derived GBM lines used (WT = wild type;
U = unmethylated; M = methylated; + =
expressed confirmed by Western blot)

| | | | | CY19A1 |
|---|---|---|---|---|
| Cell lines | Type | MGMT | IDH | expression |
| G75 | Primary | U² | WT¹ | + |
| G43 | Primary | U | WT | + |
| JHH-136 | Primary | U | WT | + |
| G76 | Primary | M³ | WT | + |

As stated previously, PARPi used for this study and LTZ have been previously studied extensively for their clinical PK properties. Elimination of the PARPi occurs primarily through metabolism through CYP3A4 (pamiparib, olaparib and veliparib) and carboxylesterases (niraparib). LTZ, on the other hand, is metabolized primarily by CYP2A6, a minor CYP450 enzyme making pharmacokinetic drug-drug interactions between LTZ and PARPi highly unlikely. As such the brain ECF PK studies were performed for each PARPi as a single agent employing the intracerebral microdialysis. The brain ECF PK of the PARPi revealed that pamiparib effectively penetrates the BBB in rats and achieves the brain ECF concentrations (C_{max,ecf}: 1.02 μM) sufficient to cover the in vitro IC₅₀. The free exposure of pamiparib in rat brain was also observed to be sufficient to cover the in vitro IC₉₀ for combination. Niraparib (C_{max,ecf}: 0.6 μM) also had adequate BBB permeability to achieve the brain ECF concentrations in the range of in vitro IC₅₀ values in combination with LTZ, but not as a single agent. On the other hand, for olaparib (C_{max,ecf}: <0.05 μM) and veliparib (C_{max,ecf}: 0.3 μM), brain ECF concentrations were found to be significantly lower than the in vitro IC₅₀ both as single agents and in combination with LTZ.

Although the efficacy of PARPi has been demonstrated in variety of cancers, most common adverse effects (AEs) of pamiparib and veliparib include grade 3/4 neutropenia, grade 3/4 thrombocytopenia, fatigue, and nausea, and no fatal AEs have been reported. On the other hand, severe AEs such as, acute myeloid leukemia, have been reported for olaparib and niraparib at higher doses. Furthermore, the predicted dose of these PARPi as a monotherapy against GBM are significantly higher than the currently approved clinical dosing regimen. In that regard, combining PARPi with LTZ may provide a feasible approach to potentiate the activity of both the agents and reduce any systemic toxicities associated with higher doses of PARPi.

### REFERENCES

1. Ostrom, Q. T., et al., CBTRUS Statistical Report: Primary Brain and Other Central Nervous System Tumors Diagnosed in the United States in 2015-2019. Neuro Oncol, 2022. 24(Suppl 5): p. v1-v95.
2. Stupp, R., et al., Effect of Tumor-Treating Fields Plus Maintenance Temozolomide vs Maintenance Temozolomide Alone on Survival in Patients With Glioblastoma: A Randomized Clinical Trial. Jama, 2017. 318(23): p. 2306-2316.
3. Wen, P. Y. and S. Kesari, Malignant gliomas in adults. N Engl J Med, 2008. 359(5): p. 492-507.
4. Geisler, J., et al., Influence of letrozole and anastrozole on total body aromatization and plasma estrogen levels in postmenopausal breast cancer patients evaluated in a randomized, cross-over study. J Clin Oncol, 2002. 20(3): p. 751-7.
5. Miller, W. R., et al., Aromatase inhibitors: are there differences between steroidal and nonsteroidal aromatase inhibitors and do they matter? Oncologist, 2008. 13(8): p. 829-37.
6. Dave, N., G. A. Gudelsky, and P. B. Desai, The pharmacokinetics ofletrozole in brain and brain tumor in rats with orthotopically implanted C6 glioma, assessed using intracerebral microdialysis. Cancer Chemother Pharmacol, 2013. 72(2): p. 349-57.
7. Karve, A. S., et al., Potentiation of temozolomide activity against glioblastoma cells by aromatase inhibitor letrozole. Cancer Chemother Pharmacol, 2022. 90(4): p. 345-356.
8. Desai, P. B., et al., A Phase 0/I Pharmacokinetic and Pharmacodynamics and Safety and Tolerability Study of Letrozole in Combination with Standard Therapy in Recurrent High-Grade Gliomas. Clinical Cancer Research, 2024. 30(10): p. 2068-2077.
9. Lord, C. J. and A. Ashworth, PARP inhibitors: Synthetic lethality in the clinic. Science, 2017. 355(6330): p. 1152-1158.
10. Ledermann, J., et al., Olaparib maintenance therapy in platinum-sensitive relapsed ovarian cancer. N Engl J Med, 2012. 366(15): p. 1382-92.
11. Robson, M., et al., Olaparib for Metastatic Breast Cancer in Patients with a Germline BRCA Mutation. N Engl J Med, 2017. 377(6): p. 523-533.
12. Mateo, J., et al., Olaparib inpatients with metastatic castration-resistant prostate cancer with DNA repair gene aberrations (TOPARP-B): a multicentre, open-label, randomised, phase 2 trial. Lancet Oncol, 2020. 21(1): p. 162-174.
13. Kizilbash, S. H., et al., Restricted Delivery of Talazoparib Across the Blood-Brain Barrier Limits the Sensitizing Effects of PARP Inhibition on Temozolomide Therapy in Glioblastoma. Mol Cancer Ther, 2017. 16(12): p. 2735-2746.
14. Wang, J., et al., Comparative efficacy, safety, and acceptability of single-agent poly (ADP-ribose) polymerase (PARP) inhibitors in BRCA-mutated HER2-negative metastatic or advanced breast cancer: a network meta-analysis. Aging (Albany NY), 2020. 13(1): p. 450-459.
15. Mateo, J., S. Carreira, and J. S. de Bono, PARP Inhibitors for Advanced Prostate Cancer: Validating Predictive Biomarkers. Eur Urol, 2019. 76(4): p. 459-460.
16. Vaubel, R. A., et al., Genomic and Phenotypic Characterization of a Broad Panel of Patient-Derived Xenografts Reflects the Diversity of Glioblastoma. Clin Cancer Res, 2020. 26(5): p. 1094-1104.
17. Binder, Z. A., et al., Establishment and Biological Characterization of a Panel of Glioblastoma Multiforme (GBM) and GBM Variant Oncosphere Cell Lines. PLoS One, 2016. 11(3): p. e0150271.
18. Hu, Y. and G. K. Smyth, ELDA: extreme limiting dilution analysis for comparing depleted and enriched populations in stem cell and other assays. J Immunol Methods, 2009. 347(1-2): p. 70-8.
19. Gadgil, S. N., et al., Preclinical Evaluation of the Efficacy of a Cyclin-dependent Kinase Inhibitor Ribociclib in Combination with Letrozole Against Patient-Derived Glioblastoma Cells. Mol Cancer Ther, 2025.
20. Chen, A., PARP inhibitors: its role in treatment of cancer. Chin J Cancer, 2011. 30(7): p. 463-71.

Although not described in detail herein, other steps which are readily interpreted from or incorporated along with the disclosed embodiments shall be included as part of the invention. The embodiments that have been described herein provide specific examples to portray inventive elements, but will not necessarily cover all possible embodiments commonly known to those skilled in the art.

What is claimed is:

1. A method comprising:
administering an aromatase inhibitor to a subject;
wherein the subject suffers from a glioma.

2. The method of claim 1, wherein the aromatase inhibitor is letrozole.

3. The method of claim 1 wherein the glioma is a malignant glioma.

4. The method of claim 3, wherein the malignant glioma is glioblastoma.

5. The method of claim 1, where the subject has been diagnosed with a glioma.

6. The method of claim 5, wherein the glioma is glioblastoma.

7. The method of claim 2, wherein the subject is also administered temozolomide.

8. The method of claim 1, further comprising administering a poly-ADP ribose polymerase inhibitor in combination with the aromatase inhibitor.

9. The method of claim 8, wherein the poly-ADP ribose polymerase inhibitor is selected from the group consisting of olaparib, pamiparib, veliparib and niraparib.

10. The method of claim 2, further comprising administering a poly-ADP ribose polymerase inhibitor in combination with letrozole.

11. The method of claim 10, wherein the poly-ADP ribose polymerase inhibitor is selected from the group consisting of olaparib, pamiparib, veliparib and niraparib.
